(19) United States
(12) Patent Application Publication
(10) Pub. No.: US 2021/0315441 A1
(43) Pub. Date: Oct. 14, 2021

(54) HYDROPHOBIC FINISH FOR MEDICAL DEVICES

(71) Applicant: Covidien LP, Mansfield, MA (US)

(72) Inventors: Douglas M. Pattison, East Hartford, CT (US); Henry E. Holsten, Hamden, CT (US)

(21) Appl. No.: 17/222,472

(22) Filed: Apr. 5, 2021

Related U.S. Application Data
(60) Provisional application No. 63/007,410, filed on Apr. 9, 2020.

(51) Int. Cl. A61B 1/00; A61B 17/34; A61B 1/313; A61B 1/018

(52) U.S. Cl. CPC A61B 1/00096; A61B 17/3421; A61B 2017/00938; A61B 1/018; A61B 1/3132

(57) ABSTRACT

A medical device has a treated surface that avoids the need for cleaning the medical device during use. In aspects, the medical device has an optical component, such as a lens. The lens has a laser treated hydrophobic surface that repels tissue and body fluids in use.

### CROSS-REFERENCE TO RELATED APPLICATION

This application claims the benefit of and priority to U.S. Provisional Patent Application No. 63/007,410, filed Apr. 9, 2020, the entire disclosure of which is incorporated by reference herein.

### TECHNICAL FIELD

The disclosure relates to a surgical apparatus for use in minimally invasive surgical procedures, such as endoscopic and/or laparoscopic procedures and, more particularly, to a hydrophobic treatment of a lens or similar optical component of the medical device.

### BACKGROUND

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy and laryngobronchoscopy, just to name a few. In these procedures, trocars are utilized for creating incisions through which the endoscopic surgery is performed. Trocar tubes or cannula devices are extended into and left in place in the abdominal wall to provide access for endoscopic surgical tools. A medical device having an optical component, such as a camera or laparoscope, is inserted through a trocar tube to permit the visual inspection and magnification of the body cavity. The surgeon can then perform diagnostic and/or therapeutic procedures at the surgical site with the aid of specialized instrumentation, such as forceps, graspers, cutters, applicators, and the like, which are designed to fit through additional cannulas.

In use, the optical component of a medical device, such as a lens of a laparoscope, can become covered by condensation, tissue, blood, other body fluids, etc. Keeping the lens of the laparoscope clean during a procedure is thus difficult, and the time needed to clean the laparoscope during the procedure can increase both the overall time of the procedure and the amount of time a patient needs to remain under anesthesia, both of which can lead to increased risk of infection and increased recovery time.

### SUMMARY

Hydrophobic treatments are applied to optical components of medical devices. The hydrophobic treatments prevent and/or reduce fouling of the surface of the optical component, so that the optical component does not need to be cleaned during a medical procedure.

In aspects, the medical device is a laparoscope. A laparoscope of the disclosure has, an elongate body having a distal portion, a proximal portion, and a lens at the distal portion, the lens having a laser treated hydrophobic surface.

In some aspects, the laser treated hydrophobic surface of the laparoscope has a water contact angle exceeding 150°. In other aspects, the laser treated hydrophobic surface of the laparoscope has a sliding angle less than 10°.

The laser treated hydrophobic surface of the laparoscope possesses at least one property selected from anti-biofouling, anti-microbial, low flow resistance, platelet anti-adhesion, or combinations thereof.

In aspects, the laser treated hydrophobic surface of the laparoscope repels body fluids, tissue, or condensation which may adhere to or form on the lens.

In other aspects, the medical device is an optical trocar. An optical trocar of the disclosure has a cannula assembly and an obturator assembly. The obturator assembly has an obturator housing in mechanical cooperation with an elongated obturator member, and an optical tunneling member at a distal portion of the obturator member, the optical tunneling member having a laser treated hydrophobic surface.

In aspects, the optical tunneling member of the optical trocar is substantially hollow to receive a distal portion of an endoscope.

In some aspects, the laser treated hydrophobic surface of the optical trocar has a water contact angle exceeding 150°. In other aspects, the laser treated hydrophobic surface of the optical trocar has a sliding angle less than 10°.

The laser treated hydrophobic surface of the optical trocar possesses at least one property selected from anti-biofouling, anti-microbial, low flow resistance, platelet anti-adhesion, or combinations thereof.

In aspects, the laser treated hydrophobic surface of the optical trocar repels body fluids, tissue, or condensation which may adhere to or form on the optical tunneling member.

In aspects, the optical tunneling member of the optical trocar is a bladeless tip configured for penetrating body tissue.

In other aspects, the optical tunneling member of the optical trocar has a configuration selected from a sharp tip, a pointed tip, a pyramidal tip, a bladed tip, a conical tip, or a tip having one or more sharp edges.

In some aspects, the optical tunneling member of the optical trocar has a radiused blunt tip.

### BRIEF DESCRIPTION OF THE DRAWINGS

Various aspects of the disclosed trocar are described herein below with reference to the drawings, wherein:

FIG. 1 is a side perspective view of a laparoscope of the disclosure; and

FIG. 2 is a perspective view of an optical trocar of the disclosure.

### DETAILED DESCRIPTION

The disclosed medical devices are described in detail with reference to the drawings, in which like reference numerals designate identical or corresponding elements in each of the several views. As used herein the term “distal” refers to that portion of the medical device, or component thereof, farther from the user, while the term “proximal” refers to that portion of the medical device, or component thereof, closer to the user.

Medical devices of the disclosure include devices inserted in a patient to provide visualization of a target site. These medical devices may be introduced into the patient using minimally invasive procedures through natural orifices, or via a device inserted through a trocar, for example, and may be adapted to provide images of the surgical site or anatomic location such as the lungs, liver, stomach, gall bladder, urinary tract, reproductive tract, and intestinal tissue, for example. Once positioned at the target site, the medical devices may provide images that enable the surgeon to more accurately diagnose and provide more effective treatment of the diseased tissue. In aspects, the medical devices may be inserted into the tissue treatment region percutaneously. In other aspects, the medical device may be introduced into the tissue treatment region endoscopically (e.g., laparoscopically and/or thoracoscopically), through small keyhole incisions via a trocar, or through a natural orifice.

The medical devices of the disclosure include an optical component, typically at a distal portion of the medical device, which permits visualization of the target site. The optical component, for example a lens, has a hydrophobic surface treatment which prevents and/or reduces fouling of the surface of the optical component, which may otherwise occur due to the presence of condensation, tissue, blood, other body fluids, etc. This avoids having to remove the medical device to clean the optical component during a surgical procedure. Other examples of the optical component may include a window (e.g., covering a lens or through which an optical sensor senses a property of the tissue or other parameter of the surgical site).

The hydrophobic surface treatment of the optical component of the medical device of the disclosure is not a film or coating thereon, but rather a treatment of the material used to form the optical component itself, so that the resulting treated surface of the optical component is hydrophobic.

The optical component of any medical device may be formed of any suitable transparent material, including glass, transparent plastics, combinations thereof, and the like.

In aspects, laser-based processing, using one or more femtosecond (fs) duration (i.e., 1-999.99 fs) laser pulses, is used to impart the hydrophobic surface to the optical component of the medical device. This laser-based processing alters the surface structure or restructures the surface of the optical component to form the hydrophobic surface, which is referred to, in aspects, as a laser treated hydrophobic surface.

The duration of the laser pulses used to form the laser treated hydrophobic surface of the optical component of the medical device is a function of the laser system used. The laser system may be a Ti:sapphire laser system generating 65 fs duration pulses at a central wavelength of 0.8 μm; however, other laser systems generating different fs pulse durations are also contemplated. Other fs duration laser systems include, e.g., a Yb-doped fiber laser such as the FPCA uJewel (available from IMRA America, Ann Arbor Mich.) dye lasers, Cr:LiSAF lasers, KrF lasers, and others within the purview of those skilled in the art.

In addition to laser pulse duration, a number of other laser parameters may be varied to obtain the desired laser treated hydrophobic surface of the optical component of the medical device. These parameters include, but are not limited to: the polarization of the laser beam (often horizontally polarized); the diameter of the spot of laser irradiation on the surface of the material used to form the optical component of the medical device (often between 100 and 1200 μm); the wavelength of the laser beam; the energy density, F (fluence), of the laser beam; the number of laser pulses (shots) applied to the material used to form the optical component of the medical device; time delay between laser pulses; the extent of overlap between multiple laser pulses (shots) applied to the particular region of the material being processed; whether the shots are applied in a vacuum or under higher pressure conditions, and others.

According to various non-limiting aspects, the fs laser has a central wavelength (lambda) of 0.8 μm. However, other wavelengths in the IR, visible, ultraviolet, infrared, THz frequency, etc., may be used.

With regard to laser pulses, processes of the disclosure may use single- and multi-pulse exposures to produce the laser treated hydrophobic surface of the optical component of the medical device. A laser “pulse”, or “shot”, refers to a single laser pulse applied using, for example, an electromechanical shutter to select a single pulse. Multi-pulse or multi-shot situations can involve more than a single shot, e.g., 2, 3, 4, 5, 6, 7, 8, 9, 10, etc. up to thousands, tens-of-thousands, or hundreds-of-thousands of shots. The exact number of pulses or shots chosen will depend upon the desired hydrophobicity for the optical component of the medical device.

The extent of overlap between shots in a multi-shot situation may be varied in order to obtained desired effects, e.g., by specifying that at least x % of the area of an additional shot or shots overlap with the first or previous shot, where x can be 1 to 100%.

As a result of shot overlap or other controllable parameters, a variable percentage of the surface of the optical component of the medical device may be altered to have the desired structure or structures and thus the desired hydrophobicity. For example, a precise scanning pattern of a laser beam across the surface of the material used to form the optical component of the medical device may be used to ensure that a variable percentage of the surface is altered to possess the desired hydrophobicity. Contemplated percentages of the surface of the optical component of the medical device to be modified are from 1% to 100%.

In some instances, the surface of the medical device may be treated under ambient air/pressure conditions to obtain the desired hydrophobicity.

The disclosure also contemplates that purified gases may be used in addition to ambient air for use in forming the laser treated hydrophobic surface of the optical component of the medical device. Inert gases may have desirable effects, thus such gases or other purified gas or mixtures of gases may be used in aspects of the disclosure.

The resulting laser treated hydrophobic surface of the optical component of the medical device may have the following properties: (i) large water contact angle, in aspects exceeding 150°, and (ii) small sliding angle)(<10° to cause water drops to easily roll off the treated surface. In some instances, the sliding angle may be the minimum angle when a liquid droplet (e.g. a droplet of distilled water or another “clean” liquid) begins to slide down on the inclined surface at room temperature. The hydrophobicity of the resulting surface provides various functionalities to the surface of the optical component of the medical device, including anti-biofouling, anti-microbial, low flow resistance, platelet anti-adhesion, combinations thereof, and the like.

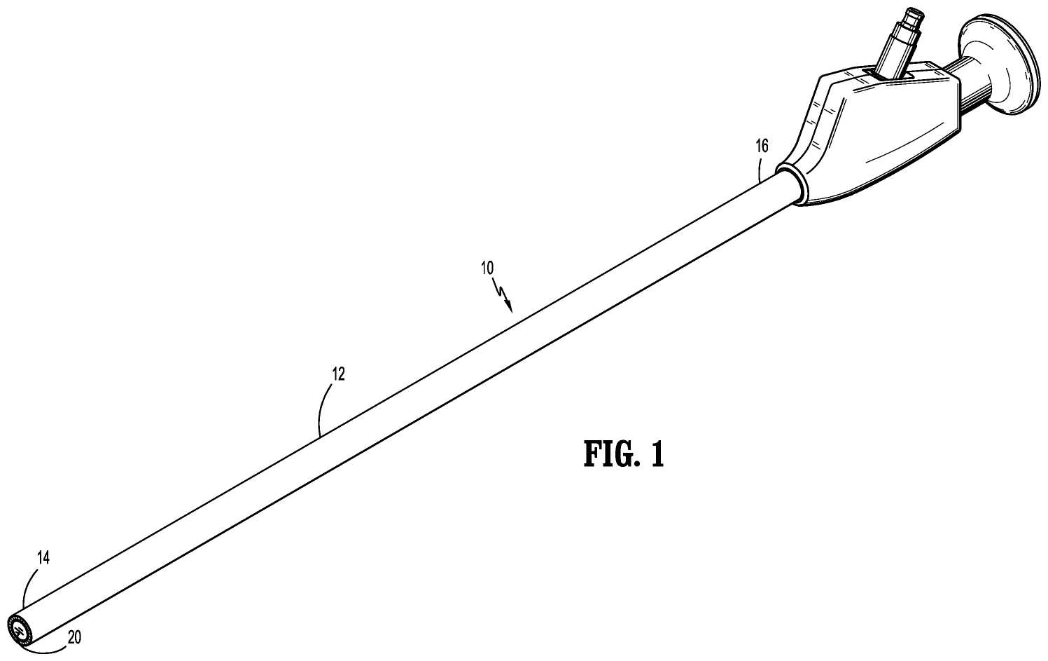

FIG. 1 illustrates an exemplary medical device of the present disclosure. FIG. 1 depicts a laparoscope 10 having an elongate body 12, a distal portion 14, and a proximal portion 16. A lens 20 is at the distal portion 14 of the laparoscope 10. The lens 20 is the optical component that has been treated as described above so that it has the laser treated hydrophobic surface which repels body fluids, tissue, and/or condensation which may otherwise adhere to or form on the lens 20 in use.

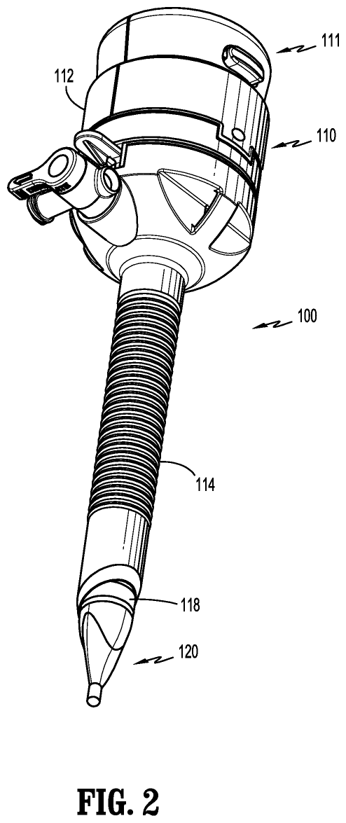

FIG. 2 illustrates an alternate medical device of the disclosure. FIG. 2 depicts an optical trocar 100 which includes an obturator assembly 111 and a cannula assembly 110. The cannula assembly 110 includes an elongated body portion 114 and is configured to at least partially receive the obturator assembly 111.

The obturator assembly 111 includes an obturator housing 112 disposed in mechanical cooperation with an elongated obturator member 118. The obturator member 118 extends distally from the obturator housing 112. The obturator member 118 includes an optical tunneling member 120 as the optical component at a distal portion of the obturator member 118 (FIG. 2). At least a portion of the optical tunneling member 120 is translucent or transparent. The term “optical tunneling member” is intended to encompass any or all of the mechanisms within the purview of the skilled artisan for blunt tip members utilized for attachment to obturator, trocar and/or cannula assemblies for separating tissue planes in a surgical procedure and for the blunt dissection of cavity linings and/or organs during a surgical procedure.

Optical tunneling member 120 may be substantially hollow to receive a distal portion of an endoscope (not shown). Improved optical characteristics of the system permit precise and accurate visual placement thereof into a body cavity. Accordingly, the access system may be suitable as an initial entry surgical access system.

In aspects, the optical tunneling member 120 is a bladeless tip configured for traversing and/or penetrating body tissue. In other aspects, the optical tunneling member 120 may be configured with for example, a sharp tip, a pointed tip, a pyramidal tip, a bladed tip, a conical tip, and/or a tip comprising one or more sharp edges or sharpened edges. In still other aspects, the optical tunneling member 120 may be a radiused blunt tip, which may be helpful for traversing an existing body orifice, and/or relatively soft or fatty tissue.

The optical tunneling member 120 has been treated as described above so that it has the laser treated hydrophobic surface which repels body fluids, tissue, and/or condensation which may otherwise adhere to or form on the optical tunneling member 120 in use.

While the above disclosure has described laparoscopes and optical trocars in detail, the disclosure is not so limited. Any medical device having an optical component to permit visualization of tissue within a patient's body may be treated as described herein so that the optical component of the medical device has the laser treated hydrophobic surface.

Any of the other components of the medical described herein may be fabricated from either metals, plastics, resins, composites or the like taking into consideration strength, durability, wearability, weight, resistance to corrosion, ease of manufacturing, cost of manufacturing, and the like.

The medical devices of the disclosure and its associated methods of use have several advantages including, for example:

enhance surgical efficiency;

maintain image quality;

avoid the need for cleaning during a procedure;

reduce surgical operation and anesthesia time; and/or

avoid the risk of infection.

It will be understood that various modifications may be made to the disclosed medical devices. Therefore, the above description should not be construed as limiting, but merely as exemplifications of aspects of the disclosure. Those skilled in the art will envision other modifications within the scope and spirit of the disclosure. For example, any and all features of one described aspect may be suitably incorporated into another aspect.

What is claimed is:

1. A laparoscope, comprising:
an elongate body having a distal portion, a proximal portion, and a lens at the distal portion, the lens having a laser treated hydrophobic surface.

2. The laparoscope of claim 1, wherein the laser treated hydrophobic surface has a water contact angle exceeding 150°.

3. The laparoscope of claim 1, wherein the laser treated hydrophobic surface has a sliding angle less than 10°.

4. The laparoscope of claim 1, wherein the laser treated hydrophobic surface possesses at least one property selected from anti-biofouling, anti-microbial, low flow resistance, platelet anti-adhesion, or combinations thereof.

5. The laparoscope of claim 1, wherein the laser treated hydrophobic surface repels body fluids, tissue, or condensation which may adhere to or form on the lens.

6. An optical trocar, comprising:
a cannula assembly; and
an obturator assembly, the obturator assembly having an obturator housing in mechanical cooperation with an elongated obturator member, and an optical tunneling member at a distal portion of the obturator member, the optical tunneling member having a laser treated hydrophobic surface.

7. The optical trocar of claim 6, wherein the optical tunneling member is substantially hollow to receive a distal portion of an endoscope.

8. The optical trocar of claim 6, wherein the laser treated hydrophobic surface has a water contact angle exceeding 150°.

9. The optical trocar of claim 6, wherein the laser treated hydrophobic surface has a sliding angle less than 10°.

10. The optical trocar of claim 6, wherein the laser treated hydrophobic surface possesses at least one property selected from anti-biofouling, anti-microbial, low flow resistance, platelet anti-adhesion, or combinations thereof.

11. The optical trocar of claim 6, wherein the laser treated hydrophobic surface repels body fluids, tissue, or condensation which may adhere to or form on the optical tunneling member.

12. The optical trocar of claim 6, wherein the optical tunneling member is a bladeless tip configured for penetrating body tissue.

13. The optical trocar of claim 6, wherein the optical tunneling member has a configuration selected from a sharp tip, a pointed tip, a pyramidal tip, a bladed tip, a conical tip, or a tip having one or more sharp edges.

14. The optical trocar of claim 6, wherein the optical tunneling member has a radiused blunt tip.
